(19) United States
(12) Patent Application Publication
(10) Pub. No.: US 2026/0035319 A1
(43) Pub. Date: Feb. 5, 2026

(54) WASTE STREAM CONVERSION TO OLEFIN MONOMER

(71) Applicant: Basell Poliolefine Italia S.r.l., Milano (IT)

(72) Inventors: Sandor Nagy, Seabrook, TX (US); Sheri E. Davis, Baytown, TX (US); Barbara Kimmich, Houston, TX (US); Daniel F. White, Ferrara (IT); William V. Knowles, Friendswood, TX (US); David S. Mannel, Humble, TX (US); Christopher D. Smith, Kingwood, TX (US); Bryan Hare, Humble, TX (US)

(73) Assignee: Basell Poliolefine Italia S.r.l., Milano (IT)

(21) Appl. No.: 19/283,436

(22) Filed: Jul. 29, 2025

Related U.S. Application Data
(60) Provisional application No. 63/677,775, filed on Jul. 31, 2024.

(51) Int. Cl. C07C 4/06; C10G 1/02

(52) U.S. Cl. CPC C07C 4/06; C10G 1/02; C10G 2300/1003; C10G 2400/20; C10G 2400/22; C10G 2400/30

(57) ABSTRACT

A process for obtaining light olefins from a hydrocarbonaceous feedstock comprising a polymer recyclate, a polyol, a biomass composition, or a combination thereof. A process comprising contacting the hydrocarbonaceous feedstock with a small pore size zeolite catalyst in a reaction zone under pyrolysis conditions to produce a product stream comprising ethylene and/or propylene and aromatics. The hydrocarbonaceous feedstock is subjected to first pyrolysis conditions in a first reaction zone to produce a first reaction product. The first reaction product is then contacted with a small pore size zeolite catalyst in a second reaction zone under second pyrolysis conditions sufficient produce a second reaction product comprising ethylene and/or propylene and less than or equal to 8 wt % aromatics, based on the weight of the product stream. A hydrocarbonaceous waste stream is pretreated to produce the hydrocarbonaceous feedstock having a reduced content of contaminants harmful to zeolite catalysts.

This application claims the benefit of priority to U.S. Provisional Application No. 63/677,775, filed on Jul. 31, 2024, which is incorporated here by reference in its entirety.

### FIELD OF THE INVENTION

The disclosure relates to processes for catalytically pyrolyzing hydrocarbonaceous waste streams into products comprising olefin monomers. Production of aromatics in such products is suppressed by use of small pore size zeolite catalysts.

### BACKGROUND OF THE INVENTION

The conversion of hydrocarbonaceous waste streams into more valuable products is desirable from the standpoints of both economic and environmental sustainability. One of the primary objectives in processing hydrocarbonaceous waste streams is the production of light olefins, such as ethylene and propylene, which serve as fundamental building blocks for a wide range of chemical products and materials.

Traditional methods for producing light olefins typically involve catalytic pyrolysis employ a mixture of hydrocarbon feed and large pore zeolites, such as ZSM-5, which are effective in cracking reactions but also produce a substantial amount of aromatics. The production of aromatics is often undesirable due to market demand, environmental regulations, and the need for additional processing steps to separate and purify these compounds.

It would be desirable to develop methods for treatment of hydrocarbonaceous waste streams that produce products having a higher ratio of olefins (e.g., ethylene, propylene, and/or butene) relative to aromatics.

### SUMMARY OF THE INVENTION

The present disclosure provides processes for catalytic pyrolysis of hydrocarbonaceous waste streams to higher value products useful as feedstocks to other processes. In some embodiments, a hydrocarbonaceous waste stream feedstock comprises a polymer recyclate, a polyol, a biomass composition, or a combination thereof.

In some embodiments, a process to obtain light olefins from a hydrocarbonaceous feedstock comprises contacting a hydrocarbonaceous feedstock with a zeolite catalyst having a pore size ranging from 0.30 nanometers to 0.50 nanometers in a reaction zone to produce a product stream comprising an olefin component and an aromatic component. The olefin component comprises ethylene, propylene, or a combination thereof. The aromatic component comprises benzene, toluene, xylene, or a combination thereof. The product stream comprises the aromatic component in an amount less than or equal to 8 wt %, based on the total weight of the product stream.

In some embodiments, a weight ratio of the olefin component to the aromatic component is greater than or equal to 2.0. In some embodiments, the reaction zone comprises a fixed catalyst bed. In some embodiments, the reaction zone comprises a fluidized catalyst bed.

In some embodiments, a process to obtain light olefins from a hydrocarbonaceous feedstock further comprises contacting a hydrocarbonaceous waste stream is contacted with a poison mitigation compound in a guard zone to produce a treated hydrocarbonaceous waste stream. The treated hydrocarbonaceous waste stream is withdrawn as the hydrocarbonaceous feed stream. The hydrocarbonaceous feed stream is then contacted with the zeolite catalyst as described above. The hydrocarbonaceous waste stream comprises the hydrocarbonaceous feedstock and a first contaminant component. The treated hydrocarbonaceous waste stream comprises the hydrocarbonaceous feedstock and a second contaminant component. The first contaminant component would deactivate the zeolite catalyst at a first deactivation rate. The second contaminant component would deactivate the zeolite catalyst at a second deactivation rate. The first deactivation rate is greater than the second deactivation rate.

In some embodiments, a process to obtain light olefins from a hydrocarbonaceous feedstock further comprises pyrolyzing a hydrocarbonaceous waste stream to produce a first pyrolysis product in a thermal pyrolysis reaction zone. The first pyrolysis product is separated into a first pyrolysis light fraction and a first pyrolysis heavy fraction in a separation reaction zone. The first pyrolysis light fraction is withdrawn as the hydrocarbonaceous feed stream. The hydrocarbonaceous feed stream is then contacted with the zeolite catalyst as described above.

In some embodiments, a process to obtain light olefins from a hydrocarbonaceous feedstock further comprises contacting a hydrocarbonaceous waste stream with a poison mitigation compound in a guard zone to produce a treated hydrocarbonaceous waste stream. The treated hydrocarbonaceous waste stream is thermally pyrolyzed to produce a first pyrolysis product in a thermal pyrolysis reaction zone. The first pyrolysis product is separated into a first pyrolysis light fraction and a first pyrolysis heavy fraction in a separation reaction zone. The first pyrolysis light fraction is withdrawn as the hydrocarbonaceous feed stream. The hydrocarbonaceous feed stream is then contacted with the zeolite catalyst as described above. The hydrocarbonaceous waste stream comprises the hydrocarbonaceous feedstock and a first contaminant component. The treated hydrocarbonaceous waste stream comprises the hydrocarbonaceous feedstock and a second contaminant component. The first contaminant component would deactivate the zeolite catalyst at a first deactivation rate. The second contaminant component would deactivate the zeolite catalyst at a second deactivation rate. The first deactivation rate is greater than the second deactivation rate.

In some embodiments, a process to obtain light olefins from a hydrocarbonaceous feedstock further comprises thermally pyrolyzing a hydrocarbonaceous waste stream to produce a first pyrolysis product in a thermal pyrolysis reaction zone. The first pyrolysis product is separated into a first pyrolysis light fraction and a first pyrolysis heavy fraction in a separation reaction zone. The first pyrolysis light fraction is contacted with a poison mitigation compound in a guard zone to produce a treated first pyrolysis light fraction. The treated first pyrolysis light fraction is withdrawn as the hydrocarbonaceous feed stream. The hydrocarbonaceous feed stream is then contacted with the zeolite catalyst as described above. The hydrocarbonaceous feed stream is then processed according to the first and second groups of embodiments. The hydrocarbonaceous waste stream comprises the hydrocarbonaceous feedstock and a first contaminant component. The treated hydrocarbonaceous waste stream comprises the hydrocarbonaceous feedstock and a second contaminant component. The first contaminant component would deactivate the zeolite catalyst at a first deactivation rate. The second contaminant component would deactivate the zeolite catalyst at a second deactivation rate. The first deactivation rate is greater than the second deactivation rate.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other catalyst compositions and/or processes for carrying out the same purposes of the present invention. It should also be realized by those skilled in the art that such equivalent constructions do not depart from the spirit and scope of the invention as set forth in the appended claims. The novel features which are believed to be characteristic of the invention, both as to its compositions and methods, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1 is a simplified flow diagram of an embodiment of the disclosed process wherein a hydrocarbonaceous waste stream is fed to a catalytic pyrolysis reaction zone followed by separation of the reaction product to recover a product stream comprising ethylene and propylene;

FIG. 1A, in conjunction with FIG. 1, is a simplified flow diagram of an embodiment of the disclosed process wherein the hydrocarbonaceous waste stream is pretreated in a guard reaction zone prior to being fed to the process shown in FIG. 1;

FIG. 1B, in conjunction with FIG. 1, is a simplified flow diagram of an embodiment of the disclosed process wherein the hydrocarbonaceous waste stream is pretreated in a thermal pyrolysis zone followed by separation of the reaction product to recover a lighter stream for feed to the process shown in FIG. 1;

FIG. 1C, in conjunction with FIG. 1, is a simplified flow diagram of an embodiment of the disclosed process wherein the hydrocarbonaceous waste stream is pretreated in a guard reaction zone prior to being fed to a thermal pyrolysis zone followed by separation of the reaction product to recover a lighter stream for feed to the process shown in FIG. 1; and

FIG. 1D, in conjunction with FIG. 1, is a simplified flow diagram of an embodiment of the disclosed process wherein the hydrocarbonaceous waste stream is pretreated in a thermal pyrolysis zone followed by separation of the reaction product to recover a lighter stream, which is further treated in a guard reaction zone prior to being fed to the process shown in FIG. 1.

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase. It must also be noted that, as used in the specification and the appended claims, the singular forms “a,” “an,” and “the” include plural references unless otherwise specified.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “char” refers to the solid residue obtained from the pyrolysis of hydrocarbonaceous feed at relatively low temperatures, typically under inert atmosphere conditions. It consists mainly of carbon along with ash and residual volatile matter. The formation of char is a critical step in the pyrolysis process, where the thermal decomposition of hydrocarbonaceous material occurs in the absence of oxygen, preventing combustion. Char is rich in carbon but also contains a significant amount of other elements like oxygen, hydrogen, and nitrogen.

As used herein, “coke” refers to the carbonaceous solid derived from the pyrolysis of hydrocarbonaceous feed at higher temperatures. The resulting material is a highly porous, carbon-rich solid that has high thermal stability and structural strength. Unlike char, coke is generally less reactive chemically due to its higher carbon purity and lower volatile content.

As used herein, the terms “hydrocarbon,” “hydrocarbons,” and “hydrocarbonaceous” do not mean materials strictly or only containing hydrogen atoms and carbon atoms. Such terms include materials that are hydrocarbonaceous in nature in that they primarily or essentially are composed of hydrogen and carbon atoms, but can contain other elements such as oxygen, sulfur, nitrogen, metals, inorganic salts, and the like, even in significant amounts.

As used herein, “propylene glycol,” in reference to the feed stream to the process disclosed herein, refers to mono-propylene glycol, di-propylene glycol, tri-propylene glycol, tetra-propylene glycol, higher polypropylene glycols, or a combination thereof.

As used herein, “poison mitigation compound” means a composition that absorbs at least a portion of the impurities or contaminants, degrades at least a portion of the impurities or contaminants. In some embodiments, a portion of the impurities or contaminants in the hydrocarbonaceous waste stream is absorbed by the poison mitigation compound to reduce the amount of impurities or contaminants that will enter the reaction zone comprising the zeolite catalyst. In some embodiments, a portion of the impurities or contaminants in the hydrocarbonaceous waste stream is degraded by the poison mitigation compound to break down a portion of the impurities or contaminants into lower molecular weight compounds that will enter the reaction zone comprising the zeolite catalyst. In some embodiments, a portion of the impurities or contaminants in the hydrocarbonaceous waste stream is degraded by the poison mitigation compound to break down a portion of the impurities or contaminants into lower molecular weight compounds and a portion of the lower molecular weight compounds are absorbed by the poison mitigation compound such that only a portion of the lower molecular weight compounds will enter the reaction zone comprising the zeolite catalyst.

As used herein, “impurities” or “contaminants,” in reference to the hydrocarbonaceous waste stream, refers to material present that can reduce the catalytic activity and/or the useful life of a zeolite catalyst. In some embodiments, impurities or contaminants comprise amines, urethane, amides, other nitrogen containing hydrocarbons, organic bases, caustic, or a combination thereof.

As used herein, “post-consumer waste” refers to a type of waste produced by the end consumer of a material stream.

As used herein, “post-industrial waste” refers to a type of waste produced during the production process of a product.

As used herein, “reaction zone” refers to a chamber sufficiently enclosed to maintain selected operating conditions within the chamber to produce a desired reaction. In some embodiments, a single vessel can contain a plurality of reaction zones.

As used herein, “waste stream” is a type of feed stream comprising material that is a byproduct of a process and/or that has been discarded as no longer useful, including but not limited to, post-consumer and post-industrial waste.

As used herein, “zeolite” refers to an aluminosilicate mineral with a microporous structure. Zeolites are, in one aspect, useful as catalysts for the processes disclosed herein. Zeolites can occur naturally or can be produced industrially.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the disclosure. As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

All concentrations herein are by weight percent (“wt %”) unless otherwise specified.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

### Catalytic Pyrolysis of Hydrocarbonaceous Waste Streams

Small pore size zeolites have been identified as potential candidates for cracking reactions due to their unique pore structures. These small pore size zeolites, such as chabazite and erionite, have been found to be less active in traditional cracking processes, which has limited their use in industrial applications. However, it has been surprisingly discovered that of small pore size zeolites have the capability to selectively produce light olefins while suppressing the formation of aromatics.

The present disclosure provides methods for processing hydrocarbonaceous waste streams by using small pore size zeolites, wherein the hydorcarbonaceous waste stream comprises a hydrocarbonaceous feedstream and a contaminant component. The contaminant component comprises one or more compounds which are poisons to a zeolite catalyst. A variety of waste stream feedstocks, including, but not limited to, polyolefins, vegetable oils, and glycols, into light olefins. By leveraging the unique properties of small pore size zeolites, selectivity towards light olefins is enhanced and the environmental impact associated with the production of aromatics is reduced. The present disclosure provides a process to obtain an olefin component, comprising light olefins (e.g., ethylene and/or propylene), from a hydrocarbonaceous feedstock.

In some embodiments, the hydrocarbonaceous feedstock comprises a polymer recyclate, a polyol, a biomass composition, or a combination thereof. The process comprises contacting the hydrocarbonaceous feedstock with a small pore size zeolite catalyst in a reaction zone under pyrolysis conditions sufficient to produce a product stream comprising ethylene and/or propylene and less than or equal to 8 wt % aromatics, based on the weight of the product stream.

As shown in the examples below, small pore size zeolites favor production of light olefins (e.g., ethylene and/or propylene). In some embodiments, the disclosed pyrolysis process using small pore size zeolites converts a variety of waste streams to produce products having a higher content of ethylene and/or propylene. In some embodiments, ratio of the combined weight percent of ethylene and propylene in the product stream to the weight percent aromatics (e.g., benzene, toluene, and xylene (BTX)) in the product stream is greater than or equal to 2.0, greater than or equal to 4.0, greater than or equal to 6.0, greater than or equal to 8.0, greater than or equal to 10.0, or greater than or equal to 2.0, greater than or equal to 14.0, or greater than or equal to 16.0.

In some embodiments, the catalytic pyrolysis reaction zone comprises a fixed catalyst bed or a fluidized catalyst bed. The cracking process can be carried out in a batch, continuous, semi-batch or semi-continuous manner using conventional reactor systems such as fixed bed, moving bed, fluidized bed and the like. Conventional catalyst regeneration techniques can also be employed. The feed is contacted with the catalyst under reaction conditions effective to form light olefins, and preferably favoring light olefins. Contacting is preferably done in the vapor phase by bringing vaporous feed into contact with the solid catalyst. The feed and/or catalyst can be preheated as desired.

In some embodiments, the reaction is performed at a temperature in the range of from about 200° C. to about 800° C., from 300° C. to 700° C., or from 400° C. to 600° C.

In some embodiments, the reaction is performed at pressures in the range of from about zero (i.e., atmospheric pressure) to about 10 MPag, from about zero to about 1 MPag, from about zero to about 100 kPag, or from about zero to about 10 kPag.

In some embodiments, the reaction is performed at a weight hourly space velocity feed rates in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹, with or without a conventional diluent. Typical diluents include, but are not limited to, steam, recycle gases, inert gases, or a combination thereof.

A feature of the fluidized catalytic cracking (FCC) process is the regeneration of the spent catalyst, which is a critical component for sustaining the continuous operation of the system. In this process, the catalyst, having been deactivated by coke deposition, is directed to a regeneration unit where it undergoes rejuvenation. This rejuvenation process involves burning off the coke in an oxygen-rich environment, thereby restoring the catalyst's activity before it is recirculated into the reactor. This regeneration cycle is imperative for maintaining the catalyst's operational efficiency and, by extension, the productivity of the FCC system. See U.S. Pat. Nos. 5,043,522 and 5,026,936, both to Leyshon et al., each incorporated by reference in their entirety herein.

In contrast to the continuous operation of the FCC system, the present disclosure provides for application of multiple batch reactors operating in parallel. This configuration allows for a quasi-continuous operation by strategically cycling reactors through different phases of operation, including production, cleaning, preparation, and catalyst regeneration. Notably, for processes necessitating catalyst regeneration, the present invention envisages a separate unit for the regeneration of spent catalysts from the batch reactors. This enables the reuse of catalysts across subsequent batches, thereby enhancing the operational efficiency and reducing the downtime typically associated with batch processes.

In some embodiments, the process further comprises subjecting the product stream to one or more separation processes to recover an olefin product stream comprising at least 80 wt % of ethylene, propylene, or a combination thereof. Such separation processes can include one or more of distillation, hydroprocessing, solvent extraction, adsorption, and adsorption. Such embodiments would include common equipment associated with the forgoing separation processes, including, but not limited to, columns, drums, vessels, heat exchangers, pumps, valves, reflux loops, and the like, the descriptions of which are omitted herein for simplicity.

The pore structure of zeolites is a defining feature that significantly influences their adsorption and catalytic properties. The pores are formed by the arrangement of silica and alumina tetrahedra in the zeolite crystal structure, creating a network of cavities and channels. These channels and cavities are accessed through openings that are defined by the number of oxygen atoms forming the ring at the entrance. The size of these openings is critical for determining the zeolite's ability to adsorb certain molecules or catalyze specific reactions, and they are typically described by the number of tetrahedral atoms (usually silicon or aluminum) that make up the ring structure of the pore opening, commonly referred to as “membered rings.”

Small pore size zeolites are characterized by 8-membered ring structures, with pore openings ranging from 0.30 or 0.35 to 0.40, 0.45, or 0.50 nanometers. These 8-membered rings create a highly selective and confined environment, making small pore size zeolites particularly effective for separating small molecules such as hydrogen, carbon dioxide, and water. The small size of the pores restricts access to larger molecules, conferring high selectivity in catalytic processes where only small reactants are desired to participate. For example, in the conversion of methanol to olefins, small pore size zeolites can selectively produce ethylene and propylene by excluding larger molecules from the reaction sites within the pores.

Medium pore zeolites typically have 10-membered ring structures, with pore diameters ranging from 0.40, 0.45, or 0.50 to 0.60 nanometers. This pore size allows for the adsorption and catalysis of medium-sized molecules, making these zeolites versatile for a wide range of applications, including the catalytic cracking of hydrocarbons to produce gasoline. The 10-membered rings offer a balance between accessibility and selectivity, enabling the processing of larger molecules than those accommodated by 8-membered rings while still providing a degree of shape selectivity that is beneficial for many catalytic processes.

Large pore zeolites, with 12-membered ring structures and pore openings greater than 0.6 nanometers, are capable of accommodating even larger molecules. These zeolites are particularly useful for applications that involve bulky molecules, such as the processing of heavy oil fractions or the synthesis of large organic compounds. The large pores facilitate the diffusion of large reactants and products, which is essential for reactions that involve sizable molecules. However, the broad accessibility also means that large pore zeolites may exhibit lower selectivity compared to their smaller pore counterparts.

The pore structure, specifically the size and shape of the openings defined by the membered rings, is crucial for the performance of zeolites in adsorption and catalysis. The 8-membered rings of small pore size zeolites offer high selectivity for small molecules, the 10-membered rings of medium pore zeolites provide a balance of selectivity and accessibility for medium-sized molecules, and the 12-membered rings of large pore zeolites allow for the processing of large molecules.

In some embodiments, a small pore size zeolite catalyst comprises a zeolite A (LTA), a chabazite (CHA), an erionite (ERI), a zeolite T (TON), a zeolite RHO (RHO), a clinoptilolite (HEU), a gmelinite (GME), levynite (LEV), a paulingite (PAU), an analcime (ANA), a bikitaite (BIK), a zeolite L (LTL), or a combination thereof. In some embodiments, silicoaluminophosphates (SAPOs) are considered small pore size zeolites as used herein, wherein SAPO-34 has a CHA-type structure and SAPO-35 has a LEV-type structure.

In some embodiments, a small pore size zeolite catalyst has a silica-to-alumina ratio in the range of from 5 to 35, from 7 to 30, or from 10 to 28. In the case of silicoaluminophosphates, the equivalent of silica to alumina ratio (SAR) is the ratio of silica to alumina plus phosphate (S/(Al+P)).

In some embodiments, a polymer recyclate comprises a polyethylene recyclate, a polypropylene recyclate, or a combination thereof. In some embodiments, the polyethylene recyclate comprises a low density polyethylene (LDPE), linear low density polyethylene (LLDPE), medium density polyethylene, high density polyethylene (HDPE), or a combination thereof.

Suitable virgin polyethylenes used in consumer products that lead to polymer recyclates include ethylene homopolymers and copolymers of units derived from ethylene and units derived from one or more of C₃-C₂₀ alpha-olefins or mixtures thereof. In some embodiments, the units derived from the one or more C₃-C₈ alpha-olefin comonomers are present in amounts up to 15 wt. 9%, based upon the total weight of the copolymer of ethylene. The ethylene homopolymers and copolymers can be produced using either Ziegler Natta catalyst, chromium-based catalyst, or single-site catalyst, e.g., metallocene catalyst. The ethylene homopolymers and copolymers can be produced using a gas phase process, high pressure process, slurry process, or solution process. Ethylene homopolymers and ethylene-C₃-C₈ alpha-olefin copolymers include very low density polyethylene (VLDPE), low density polyethylene (LDPE), linear low density polyethylene (LLDPE), medium density polyethylene (MDPE) and high density polyethylene (HDPE). VLDPE is defined as having a density of 0.860 to 0.910 g/cm³, as measured by ASTM D-1505 “Column Method.” LDPE and LLDPE are defined as having densities in the range of from 0.90 to 0.930 g/cm³. MDPE is defined as having a density of 0.925 to 0.940 g/cm³. HDPE is defined as having a density of at least 0.945 g/cm³, preferably from 0.945 to 0.969 g/cm³. The ethylene homopolymers and copolymers preferably have melt indexes (MIs), as measured by ASTM D-1238, condition 190° C./2.16 kg, from 0.01 to 400 dg/min., preferably, from 0.1 to 200 dg/min., more preferably from 1 to 100 dg/min.

Suitable virgin polypropylenes used in consumer products that lead to polymer recyclates include propylene homopolymers and copolymers, including plastomers, having of units derived from propylene and units derived one or more of ethylene and C₄-C₂₀ alpha-olefins or mixtures thereof. Preferably, the units derived from one or more of ethylene and C₄-C₁₀ alpha-olefin comonomers are present in amounts up to 35 wt. %, based upon the total weight of the copolymer of propylene. The propylene homopolymers and copolymers can be produced using either Ziegler Natta or single-site catalysts, e.g., metallocene catalysts. The propylene homopolymers and copolymers can be produced using a gas phase process, slurry process, or solution process. In some embodiments, when the propylene polymer is a copolymer, it preferably contains 2 to 6 wt. %, based upon the total weight of the copolymer, of ethylene derived units as a comonomer.

In some embodiments, a polyol comprises one or more polyols. In some embodiments, one or more polyols comprise dihydric alcohols (glycols), trihydric alcohols (triols), tetrahydric alcohols, pentahydric alcohols, hexahydric alcohols, polyhydric alcohols (more than six hydroxyl groups), or a combination thereof. In some embodiments, dihydric alcohols (glycols) comprise ethylene glycol, propylene glycol, butylene glycol, 1,4-butanediol, 1,3-propanediol, 1,2-propanediol (propylene glycol), 2,3-butanediol, hexylene glycol, diethylene glycol, dipropylene glycol, di-propylene glycol, tri-propylene glycol, tetra-propylene glycol, or any combination thereof. In some embodiments, trihydric alcohols (triols) comprise glycerol (glycerin), trimethylolpropane, 1,2,3-propanetriol (glycerol), 1,2,4-butanetriol, 1,2,6-hexanetriol, or any combination thereof. In some embodiments, tetrahydric alcohols comprise erythritol, pentaerythritol, threitol, erythrulose, or any combination thereof. In some embodiments, pentahydric alcohols comprise xylitol, arabitol, ribitol,, or any combination thereof. In some embodiments, hexahydric alcohols comprise mannitol, sorbitol, iditol, galactitol, fucitol, or any combination thereof. In some embodiments, polyhydric alcohols comprise maltitol, lactitol, isomalt, polyethylene glycol (PEG), or any combination thereof.

In some embodiments, a biomass composition comprises a glyceride composition, a cellulose composition, a lipid composition, a carbohydrate composition, or a combination thereof. In some embodiments, the glyceride composition comprises a triglyceride. In some embodiments, the triglyceride comprises a vegetable oil. In some embodiments, the vegetable oil comprises olive oil or soybean oil.

### Pretreatment of Hydrocarbonaceous Waste Streams

In some embodiments, the feed comprising hydrocarbonaceous waste streams comprising is pretreated prior to catalytic pyrolysis. In some embodiments, the hydrocarbonaceous waste streams comprise a polymer recyclate, a polyol, a biomass composition, or a combination thereof. In some embodiments, the feed is treated in a guard reaction zone, a thermal pyrolysis reaction zone, or a combination thereof.

### Guard Reaction Zone

In some embodiments, pretreatment of the feed stream comprises adding the feed to a guard reaction zone to form a first reaction product suitable as a feed stream to the catalytic pyrolysis reaction zone as described elsewhere herein. In some embodiments, the hydrocarbonaceous feed stream comprises impurities or contaminants harmful to the catalytic activity of the zeolite catalyst utilized in the catalytic pyrolysis reaction zone. In some embodiments, such impurities or contaminants comprise amines, urethane, amides, other nitrogen containing hydrocarbons, organic bases, caustic, or a combination thereof. In some embodiments, the guard reaction zone comprises a reactive bed comprising generic absorbents, clays, diatomites, activated carbon, or a combination thereof. In some embodiments, the guard reaction zone can be operated at a pressure in the range of from 0 psig (0 kPag) to 10 psig (69 kPag), a temperature in the range of from 20° C. to 30° C., and/or. a weight hourly space velocity in the range of from 0.1 hr⁻¹ to 100 hr⁻¹

### Thermal Pyrolysis

In some embodiments, pretreatment of the feed stream comprises subjecting the feed to a thermal pyrolysis reaction zone to form a first reaction product having a light fraction suitable as a feed stream to the catalytic pyrolysis reaction zone as described elsewhere herein. The hydrocarbonaceous feed is reacted under depolymerization conditions to form a first pyrolysis product. The first depolymerization product is withdrawn from the first pyrolysis reaction zone and sent to one or more separation processes to recover a light fraction as a feed stream to the catalytic pyrolysis reaction zone.

In some embodiments, the thermal pyrolysis reaction conditions comprise a temperature in the range of from 250° C. to 450° C., from 280° C. to 420° C., or from 280° C. to 350° C. and a pressure in the range of from 5 kPa (absolute) to 10 MPag, from 13.3 kPa (absolute) to 1 MPa, from atmospheric pressure to 100 kPa, or from atmospheric pressure to about 10 kPag. In some embodiments, pretreatment of the hydrocarbonaceous feed in the thermal pyrolysis reaction zone further comprises the addition of a poison mitigation compound to promote removal of impurities or contaminants harmful to a zeolite catalyst.

### Poison Mitigation

In some embodiments, the pretreatment of the hydrocarbonaceous feed further comprises adding a poison mitigation compound to the thermal pyrolysis reaction zone. In some embodiments, the poison mitigation compound comprises CaO, CaCO₃, Ca(OH)₂, MgO, MgCO₃, Mg(OH)₂, KO₂, K₂CO₃, KOH, NaO₂, Na₂CO₃, NaOH, Zr(HPO₄)₂, a clay, an activated clay, a coke, an activated carbon, a diatomite, or a combination thereof, wherein in further embodiments the clay comprises a smectite, a vermiculite, Fuller's earth, or a combination thereof. In some embodiments, the poison mitigation compound is added in an amount less than or equal to 20 wt %, from 1 wt % to 10 wt %, or from 2 wt % to 5 wt %, based on the total weight of the hydrocarbonaceous feed and the poison mitigation compound.

### Combination Feed Pretreatment

In some embodiments, the method for pretreating the hydrocarbonaceous feed comprises a guard reaction zone and a thermal pyrolysis reaction zone.

In some embodiments, the hydrocarbonaceous stream is fed to a guard reaction zone to produce a first treated product stream. The first treated product stream, having a reduced content of impurities or contaminants harmful to zeolite catalyst, is fed to a thermal pyrolysis reaction zone to produce a first pyrolysis product stream. The first pyrolysis product is withdrawn from the first pyrolysis reaction zone and sent to one or more separation processes to recover a light fraction as a feed stream to the catalytic pyrolysis reaction zone.

In some embodiments, the hydrocarbonaceous stream is fed to a thermal pyrolysis reaction zone to produce a first pyrolysis product stream. The first pyrolysis product is withdrawn from the first pyrolysis reaction zone and sent to one or more separation processes to recover a light fraction, which is sent to a guard reaction zone to produce a first treated product stream. The first treated product stream, having a reduced content of impurities or contaminants harmful to zeolite catalyst, is fed to the catalytic pyrolysis reaction zone.

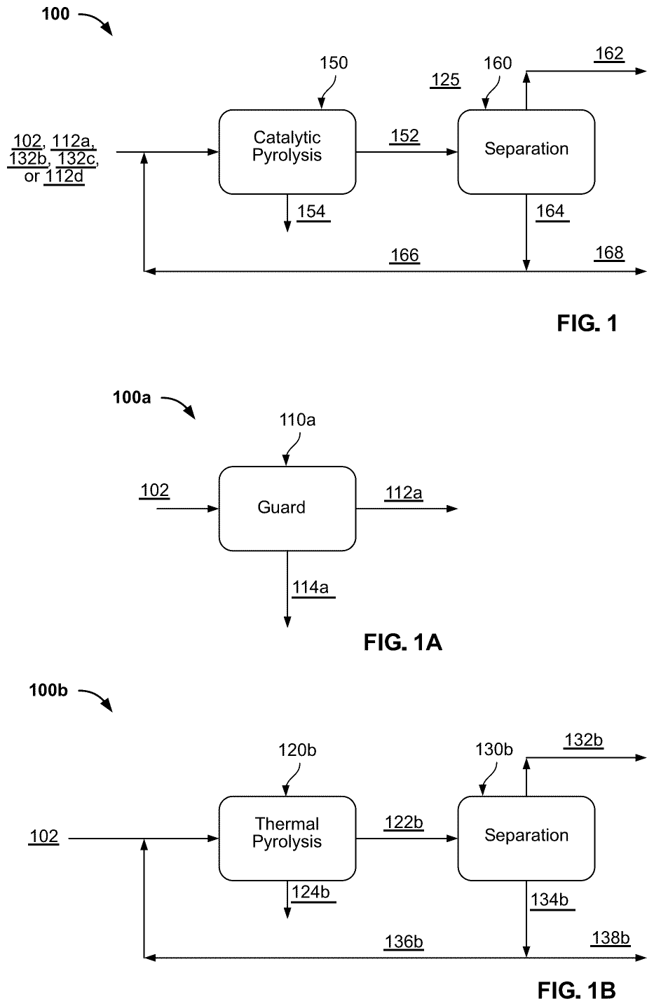

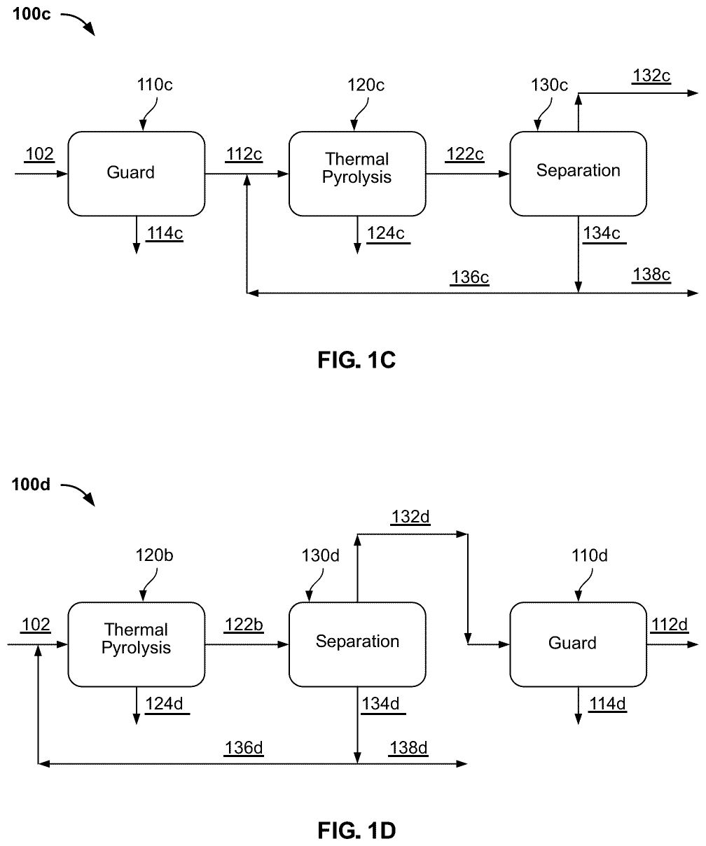

FIG. 1 shows a simplified flow diagram of a catalytic pyrolysis process 100 as disclosed herein. A hydrocarbonaceous feed stream 102 is fed to a catalytic pyrolysis reaction zone 150 to produce a depolymerization product stream 152 and char and/or coke 154. The depolymerization product stream 152 is fed to one or more separation processes 160 to produce a light fraction 162 comprising ethylene and/or propylene and a heavy fraction 164. The heavy fraction 164 is recycled as additional feed to the catalytic pyrolysis reaction zone 150 via stream 166, sent to other processing units via stream 168, or a combination thereof.

FIG. 1A shows a simplified flow diagram of an embodiment of a pretreatment process 100a for pretreatment of hydrocarbonaceous waste stream 102. A hydrocarbonaceous feed stream 102 (comprising a hydrocarbonaceous feed stream and a first contaminant component) is fed to a guard reaction zone 110a to produce a treated hydrocarbonaceous waste stream 112a (comprising a hydrocarbonaceous feed stream and a second contaminant component) and removed impurities or contaminants 114a. The treated hydrocarbonaceous waste stream 112a is then fed to the catalytic pyrolysis zone 150 in the catalytic pyrolysis process 100 as shown in FIG. 1.

FIG. 1B shows a simplified flow diagram of an embodiment of a pretreatment process 100b for pretreatment of hydrocarbonaceous waste stream to catalytic pyrolysis process 100. A hydrocarbonaceous waste stream 102 (comprising a hydrocarbonaceous feed stream and a first contaminant component) is fed to a thermal pyrolysis zone 120b to produce a first pyrolysis product 122b and char and/or coke 124b. The first pyrolysis product stream 122b is fed to one or more separation processes 130b to produce a light fraction 132b and a heavy fraction 134b.

The light fraction 132b is then fed to the catalytic pyrolysis zone 150 in the catalytic pyrolysis process 100 as shown in FIG. 1. The heavy fraction 134b is recycled as additional feed to the thermal pyrolysis reaction zone 120b via stream 136b, sent to other processing units via stream 138b, or a combination thereof.

FIG. 1C shows a simplified flow diagram of an embodiment of a pretreatment process 100c for pretreatment of hydrocarbonaceous waste stream to catalytic pyrolysis process 100. A hydrocarbonaceous feed stream 102 (comprising a hydrocarbonaceous feed stream and a first contaminant component) is fed to a guard reaction zone 110c to produce a treated feed stream 112c and removed impurities or contaminants 114c. The treated feed stream 112a is then fed to a thermal pyrolysis zone 120c to produce a first pyrolysis product 122c and char and/or coke 124c. The first pyrolysis product stream 122c is fed to one or more separation processes 130c to produce a light fraction 132c and a heavy fraction 134c.

The light fraction 132c is then fed to the catalytic pyrolysis zone 150 in the catalytic pyrolysis process 100 as shown in FIG. 1. The heavy fraction 134c is recycled as additional feed to the thermal pyrolysis reaction zone 120c via stream 136c, sent to other processing units via stream 138c, or a combination thereof.

FIG. 1D shows a simplified flow diagram of an embodiment of a pretreatment process 100d for pretreatment of hydrocarbonaceous waste stream to catalytic pyrolysis process 100. A hydrocarbonaceous feed stream 102 (comprising a hydrocarbonaceous feed stream and a first contaminant component) is fed to a thermal pyrolysis zone 120d to produce a first pyrolysis product 122d and char and/or coke 124d. The first pyrolysis product stream 122d is fed to one or more separation processes 130d to produce a light fraction 132d and a heavy fraction 134d.

The light fraction 132d is then fed to a guard reaction zone 110d to produce a treated feed stream 112d and removed impurities or contaminants 114d. The treated feed stream 112d is then fed to the catalytic pyrolysis zone 150 in the catalytic pyrolysis process 100 as shown in FIG. 1. The heavy fraction 134d is recycled as additional feed to the thermal pyrolysis reaction zone 120d via stream 136d, sent to other processing units via stream 138d, or a combination thereof.

### Certain Embodiments

In a first group of embodiments, a process comprises contacting a hydrocarbonaceous feedstock with a zeolite catalyst having a pore size ranging from 0.30 or 0.35 nanometers to 0.40, 0.45, or 0.50 nanometers in a reaction zone to produce a product stream comprising an olefin component and an aromatic component. The olefin component comprises ethylene, propylene, or a combination thereof. The aromatic component comprises benzene, toluene, xylene, or a combination thereof. The product stream comprises the aromatic component in an amount less than or equal to 8 wt %, less than or equal to 6.0 wt % aromatics, less than or equal to 4.0 wt % aromatics, less than or equal to 3.5 wt % aromatics, less than or equal to 3.0 wt % aromatics, less than or equal to 2.5 wt % aromatics, or less than or equal to 2.0 wt % aromatics, based on the total weight of the product stream.

In a second group of embodiments, the process of any one of the embodiments in the first group of embodiments is further described by one or more of the following:
a) a ratio of the weight ratio of the olefin component to the aromatic component is greater than or equal to 2.0, greater than or equal to 4.0, greater than or equal to 6.0, greater than or equal to 8.0, greater than or equal to 10.0, or greater than or equal to 2.0, greater than or equal to 14.0, or greater than or equal to 16.0;
b) the reaction zone comprises a fixed catalyst bed or a fluidized catalyst bed.
c) the process further comprises subjecting the product stream to one or more separation processes to recover an olefin product stream comprising at least 80 wt %, at least 85 wt %, at least 90 wt %, and at least 95 wt % of the olefin component;
d) the contacting step is performed at:
   i) a temperature in the range of from about 200° C. to about 800° C., from 300° C. to 700° C., or from 400° C. to 600° C.;
ii) a pressure in the range of from 5 kPa (absolute) to 10 MPag, from 13.3 kPa (absolute) to 1 MPa, from atmospheric pressure to 100 kPa, or from atmospheric pressure to about 10 kPag; or
   iii) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
   iv) a combination thereof;
e) the small pore size zeolite catalyst:
   i) comprises 8-member ring pores;
   ii) catalyst comprises a zeolite A (LTA), a chabazite (CHA), an erionite (ERI), a zeolite T (TON), a zeolite RHO (RHO), a clinoptilolite (HEU), a gmelinite (GME), levynite (LEV), a paulingite (PAU), an analcime (ANA), a bikitaite (BIK), a zeolite L (LTL), or a combination thereof;
   iii) has a silica to alumina ratio (SAR) in the range of from 5 to 35, from 7 to 30, or from 10 to 28; or
   iv) a combination thereof;
f) the hydrocarbonaceous feedstock comprises a polymer recyclate, wherein in further embodiments, the polymer recyclate comprises polyethylene recyclate, polypropylene recyclate, or a combination thereof;
g) the hydrocarbonaceous feedstock comprises one or more polyols, wherein in further embodiments, the one or more polyols comprise dihydric alcohols (glycols), trihydric alcohols (triols), tetrahydric alcohols, pentahydric alcohols, hexahydric alcohols, polyhydric alcohols (more than six hydroxyl groups), or a combination thereof; and
h) the hydrocarbonaceous feedstock comprises a biomass composition, wherein in further embodiments, the biomass composition comprises a glyceride composition, a cellulose composition, a lipid composition, a carbohydrate composition, or a combination thereof, wherein in further embodiments, the glyceride composition comprises a triglyceride, wherein in yet further embodiments, the triglyceride comprises a vegetable oil, wherein in yet further embodiments, the vegetable oil comprises olive oil or soybean oil.

In a third group of embodiments, a process to obtain light olefins from a hydrocarbonaceous feedstock further comprises contacting a hydrocarbonaceous waste stream with a poison mitigation compound in a guard zone to produce a treated hydrocarbonaceous waste stream. The treated hydrocarbonaceous waste stream is withdrawn as the hydrocarbonaceous feed stream. The hydrocarbonaceous feed stream is then processed according to the first and second groups of embodiments. The hydrocarbonaceous waste stream comprises the hydrocarbonaceous feedstock and a first contaminant component. The treated hydrocarbonaceous waste stream comprises the hydrocarbonaceous feedstock and a second contaminant component. The first contaminant component would deactivate the zeolite catalyst at a first deactivation rate. The second contaminant component would deactivate the zeolite catalyst at a second deactivation rate. The first deactivation rate is greater than the second deactivation rate.

In a fourth group of embodiments, the process of any one of the embodiments in the third group of embodiments can be further characterized by one of more of:
a) the guard zone is operated at:
   i) a temperature in the range of from 20° C. to 30° C.;
   ii) a pressure in the range of from 0 kPag (absolute) to 69 kPag;
   iii) a weight hourly space velocity in the range of from 0.1 hr⁻¹ to 100 hr⁻¹, from 0.5 hr⁻ to 60 hr⁻¹, from 0.8 hr⁻¹ to 25 hr⁻¹, from 1.0 hr⁻¹ to 25 hr⁻¹, or from 3.0 hr⁻¹ to 25 hr⁻¹; or
   iv) a combination thereof;
b) the guard zone comprises a fixed bed or a fluidized bed comprising the poison mitigation compound;
c) the poison mitigation compound absorbs at least a portion of the first contaminant component, degrades at least a portion of the first contaminant component, or a combination thereof; and
d) the poison mitigation compound comprises CaO, CaCO₃, Ca(OH)₂, MgO, MgCO₃, Mg(OH)₂, KO₂, K₂CO₃, KOH, NaO₂, Na₂CO₃, NaOH, Zr(HPO₄)₂, a clay, an activated clay, a coke, an activated carbon, a diatomite, or a combination thereof, wherein in further embodiments the clay comprises a smectite, a vermiculite, Fuller's earth, or a combination thereof.

In a fifth group of embodiments, a process to obtain light olefins from a hydrocarbonaceous feedstock further comprises pyrolyzing a hydrocarbonaceous waste stream to produce a first pyrolysis product in a thermal pyrolysis reaction zone. The first pyrolysis product is separated into a first pyrolysis light fraction and a first pyrolysis heavy fraction in a separation reaction zone. The first pyrolysis light fraction is withdrawn as the hydrocarbonaceous feed stream. The hydrocarbonaceous feed stream is then processed according to the first and second groups of embodiments.

In a sixth group of embodiments, the process of any one of the embodiments in the fifth group of embodiments can be further characterized by one of more of:
a) the process further comprises:
   i) adding at least a portion of the first pyrolysis heavy fraction as additional feed to the thermal pyrolysis reaction zone;
   ii) removing at least a portion of the first pyrolysis heavy fraction from the process; or
   iii) a combination thereof; and
b) the thermal pyrolysis reaction zone is operated at:
   i) a temperature in the range of from 250° C. to 450° C., from 280° C. to 420° C., or from 280° C. to 350° C.;
   ii) a pressure in the range of from 5 kPa (absolute) to 10 MPag, from 13.3 kPa (absolute) to 1 MPa, from atmospheric pressure to 100 kPa, or from atmospheric pressure to about 10 kPag;
   iii) a combination thereof.

In a seventh group of embodiments, a process to obtain light olefins from a hydrocarbonaceous feedstock further comprises contacting a hydrocarbonaceous waste stream with a poison mitigation compound in a guard zone to produce a treated hydrocarbonaceous waste stream. The treated hydrocarbonaceous waste stream is thermally pyrolyzed to produce a first pyrolysis product in a thermal pyrolysis reaction zone. The first pyrolysis product is separated into a first pyrolysis light fraction and a first pyrolysis heavy fraction in a separation reaction zone. The first pyrolysis light fraction is withdrawn as the hydrocarbonaceous feed stream. The hydrocarbonaceous feed stream is then processed according to the first and second groups of embodiments. The hydrocarbonaceous waste stream comprises the hydrocarbonaceous feedstock and a first contaminant component. The treated hydrocarbonaceous waste stream comprises the hydrocarbonaceous feedstock and a second contaminant component. The first contaminant component would deactivate the zeolite catalyst at a first deactivation rate. The second contaminant component would deactivate the zeolite catalyst at a second deactivation rate. The first deactivation rate is greater than the second deactivation rate.

In an eighth group of embodiments, the process of any one of the embodiments in the seventh group of embodiments can be further characterized by one of more of:
a) the process further comprises:
   i) adding at least a portion of the first pyrolysis heavy fraction as additional feed to the thermal pyrolysis reaction zone;
   ii) removing at least a portion of the first pyrolysis heavy fraction from the process; or
   iii) a combination thereof;
b) the guard zone is operated at:
   i) a temperature in the range of from 20° C. to 30° C.;
   ii) a pressure in the range of from 0 kPag (absolute) to 69 kPag;
   iii) a weight hourly space velocity in the range of from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr 1 to 25 hr⁻¹; or
   iv) a combination thereof;
c) the thermal pyrolysis reaction zone is operated at:
   i) a temperature in the range of from 250° C. to 450° C., from 280° C. to 420° C., or from 280° C. to 350° C.;
   ii) a pressure in the range of from 5 kPa (absolute) to 10 MPag, from 13.3 kPa (absolute) to 1 MPa, from atmospheric pressure to 100 kPa, or from atmospheric pressure to about 10 kPag;
   iii) a combination thereof;
d) the guard zone comprises a fixed bed or a fluidized bed comprising the poison mitigation compound;
e) the poison mitigation compound absorbs at least a portion of the first contaminant component, degrades at least a portion of the first contaminant component, or a combination thereof; and
f) the poison mitigation compound comprises CaO, CaCO₃, Ca(OH)₂, MgO, MgCO₃, Mg(OH)₂, KO₂, K₂CO₃, KOH, NaO₂, Na₂CO₃, NaOH, Zr(HPO₄)₂, a clay, an activated clay, a coke, an activated carbon, a diatomite, or a combination thereof, wherein in further embodiments the clay comprises a smectite, a vermiculite, Fuller's earth, or a combination thereof.

In a ninth group of embodiments, a process to obtain light olefins from a hydrocarbonaceous feedstock further comprises thermally pyrolyzing a hydrocarbonaceous waste stream to produce a first pyrolysis product in a thermal pyrolysis reaction zone. The first pyrolysis product is separated into a first pyrolysis light fraction and a first pyrolysis heavy fraction in a separation reaction zone. The first pyrolysis light fraction is contacted with a poison mitigation compound in a guard zone to produce a treated first pyrolysis light fraction. The treated first pyrolysis light fraction is withdrawn as the hydrocarbonaceous feed stream. The hydrocarbonaceous feed stream is then processed according to the first and second groups of embodiments. The hydrocarbonaceous waste stream comprises the hydrocarbonaceous feedstock and a first contaminant component. The treated hydrocarbonaceous waste stream comprises the hydrocarbonaceous feedstock and a second contaminant component. The first contaminant component would deactivate the zeolite catalyst at a first deactivation rate. The second contaminant component would deactivate the zeolite catalyst at a second deactivation rate. The first deactivation rate is greater than the second deactivation rate.

In a tenth group of embodiments, the process of any one of the embodiments in the ninth group of embodiments can be further characterized by one of more of:
a) the process further comprises:
   i) adding at least a portion of the first pyrolysis heavy fraction as additional feed to the thermal pyrolysis reaction zone;
   ii) removing at least a portion of the first pyrolysis heavy fraction from the process; or
   iii) a combination thereof;
b) the thermal pyrolysis reaction zone is operated at:
   i) a temperature in the range of from 250° C. to 450° C., from 280° C. to 420° C., or from 280° C. to 350° C.;
   ii) a pressure in the range of from 5 kPa (absolute) to 10 MPag, from 13.3 kPa (absolute) to 1 MPa, from atmospheric pressure to 100 kPa, or from atmospheric pressure to about 10 kPag;
   iii) a combination thereof;
c) the guard zone is operated at:
   i) a temperature in the range of from 20° C. to 30° C.;
   ii) a pressure in the range of from 0 kPag (absolute) to 69 kPag;
   iii) a weight hourly space velocity in the range of from from 0.1 hr⁻¹ to 100 hr⁻¹, from 0.5 hr⁻¹ to 60 hr⁻¹, from 0.8 hr⁻¹ to 25 hr⁻¹, from 1.0 hr⁻¹ to 25 hr⁻¹, or from 3.0 hr 1 to 25 hr⁻¹; or
   iv) a combination thereof;
d) the guard zone comprises a fixed bed or a fluidized bed comprising the poison mitigation compound;
e) the poison mitigation compound absorbs at least a portion of the first contaminant component, degrades at least a portion of the first contaminant component, or a combination thereof; and
f) the poison mitigation compound comprises CaO, CaCO₃, Ca(OH)₂, MgO, MgCO₃, Mg(OH)₂, KO₂, K₂CO₃, KOH, NaO₂, Na₂CO₃, NaOH, Zr(HPO₄)₂, a clay, an activated clay, a coke, an activated carbon, a diatomite, or a combination thereof, wherein in further embodiments the clay comprises a smectite, a vermiculite, Fuller's earth, or a combination thereof.

The presently disclosed methods for conversion of hydrocarbonaceous waste streams to ethylene and/or propylene are exemplified with respect to the examples below. These examples are included to demonstrate embodiments of the appended claims. However, these are exemplary only, and the invention can be broadly applied to any combination of hydrocarbonaceous feed disclosed zeolite catalysts. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### Examples

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Raw Materials

Conversion of various hydrocarbonaceous waste streams to light olefins was demonstrated by reacting various propylene polyols (“PPOs”) with various zeolite catalysts as shown in Table 1 below. The examples utilizing small pore size zeolites show a trend of producing ethylene, propylene, and C₄ hydrocarbons, while minimizing conversion to aromatics (benzene, toluene, xylene (BTX)).

TABLE 1

| | Zeolite | | | Available |
|---|---|---|---|---|
| Label | Catalyst | Grade | SAR | from |
| BEA-1 | Zeolite Beta | Beta CP811E-75 CY (1.6) | 75 | Zeolyst International |
| BEA-2 | Zeolite Beta | Beta CP 811 TL | 40 | Zeolyst International |
| BEA-3 | Zeolite Beta | Beta CP7119 | 26 | Zeolyst International |
| BEA-4 | Zeolite Beta | HCZB 25 | 25 | Zeolyst International |
| BEA-5 | Zeolite Beta | HCZB 150 | 150 | Zeolyst International |
| CHA-1 | Chabazite | CHA-28 | 28 | Eurocat |
| CHA-2 | Chabazite | CHA 30 | 30 | Eurocat |
| CHA-3 | Chabazite | HCZC 13 | 13 | Clariant |
| ERI-1 | Erionite | HCZE 7 | 7 | Clariant |
| MFI-1 | ZSM-5 | CBV 3014H | 30 | Zeolyst International |
| MFI-2 | ZSM-5 | CBV 3014E CY (1.6) | 30 | Zeolyst International |
| MFI-3 | ZSM-5 | CBV 3024E | 30 | Zeolyst International |
| MFI-6 | ZSM-5 | CBV 2314 | 23 | Zeolyst International |
| MOR-1 | Mordenite | Mordenite | — | Clariant |
| MOR-2 | Mordenite | HCZM 20 | 20 | Clariant |
| MOR-3 | Mordenite | CBV 21A (NH4 MOR) | — | Zeolyst International |
| Y-1 | Zeolite Y | H-USY CFG-1 | 5.2 | Zeolyst International |
| Y-2 | Zeolite Y | CFG-1 CY (1.6) | 5.2 | Zeolyst International |
| Y-3 | Zeolite Y | CBV 720 CY (1.6) | 7 | Zeolyst International |

### Waste Stream Conversion to Ethylene/Propylene

In each of Examples 1-76, approximately 0.5-1 mg of a waste stream proxy material and ˜50 mg of catalyst was added to a Frontier Lab Tandem Micro-Reactor system attached to an Agilent GC/MS with a flow of 200 ml/min of helium through the headspace above the polymer recyclate. Each sample was heated to 500 or 550° C. for 1 minute. The pyrolysate gas passed through a quartz tube containing catalyst maintained at a specified cracking temperature. Reaction product gas was cryo-trapped at the head of the GC/MS column for subsequent separation and identification.

### Determination of Zeolite Pore Size

Zeolite pore size and pore size distribution obtained on Jul. 31, 2024 from the International Zeolite Association's Database of Zeolite Structures available at https://www.iza-structure.org/databases/.

### Determination of Catalytic Pyrolysis Reaction Product

The composition of the reaction product was measured with Pyrolysis Gas Chromatography-Mass Spectrometry (Pyrolysis-GC/MS-FID) analysis using a Rx-3050TR Tandem micro-Reactor, Frontier labs, Fukushima, JP; and an Agilent 8890 GC equipped with a flame ionization detector (FID) and 5977B MSD and UA1 (30m×250 μm×2 μm) GC column, Agilent Technologies, Santa Clara, California. The GC is also equipped with a splitter to split the column effluent between the FID and MSD.

GC/MS, or gas chromatography-mass spectrometry, was used to identify the composition of the reaction product stream. Of particular interest are the ethane and ethylene (C₂), propane and propylene (C₃), mixed butanes and butenes (C₄), and benzene/toluene/xylenes (BTX). The quantification was done using the FID signal from each experiment.

### Examples 1-25-HDPE

Table 2 shows the experimental results using the listed zeolite catalysts to degrade HDPE at the listed cracking temperature (T_{cr}). Petrothene™ LM600700 HDPE, available from LyondellBasell Industries, was used as a proxy for HDPE recyclate. The products of catalytic pyrolysis of HDPE samples are reported as weight percentages of ethylene (C₂⁼), propylene (C₃⁼), mixed butanes and butenes (C₄), and benzene/toluene/xylene (BTX). Selectivity sum is the combined total amount of forgoing products as a weight percent of all pyrolysis products. Other products reports the composition of the balance of the pyrolysis product over the selectivity sum.

Inventive Examples 1˜4 show that small pore sized zeolites CHA-1, CHA-2, CHA-3, and ERI-1 produce a higher ratio of light olefins (C₂+C₃⁼) to aromatics BTX than comparative Examples 5-25 using medium or large pore zeolites.

TABLE 2
High-Density Polyethylene (HDPE)

| | | | Selectivity (wt %) | | | | | | Waste/ | | |
|---|---|---|---|---|---|---|---|---|---|---|---|
| | Zeolite | T_{cr} | | | | | Sel. | Other | Cat | (C₂⁼ + C₃⁼) | (C₂⁼to C₄) |
| Ex. | Catalyst | (° C.) | C₂⁼ | C₃⁼ | C₄ | BTX | Sum. | products¹ | (m/m) | BTX | BTX |
| 1 | CHA-3 | 500 | 6 | 41 | 20 | 2.1 | 69 | — | 1.7E−02 | 22.2 | 31.5 |
| 2 | ERI-1 | 500 | 8 | 38 | 26 | 2.5 | 75 | C₅-C₂₀ | 1.6E−02 | 18.3 | 28.8 |
| 3 | CHA-2 | 500 | 9 | 31 | 20 | 2.2 | 61 | C₅-C₂₀ | 1.8E−02 | 17.6 | 26.4 |
| 4 | CHA-1 | 500 | 5 | 25 | 21 | 2.1 | 53 | C₅-C₁₆ | 6.4E−02 | 14.2 | 24.6 |
| 5 | MOR-2 | 500 | 7 | 37 | 31 | 3.6 | 78 | C₅-C₁₂ | 1.8E−02 | 12.1 | 20.8 |
| 6 | MFI-2 | 500 | 20 | 55 | 19 | 6.6 | 100 | — | 1.4E−02 | 11.3 | 14.2 |
| 7 | BEA-5 | 500 | 4 | 32 | 32 | 3.3 | 71 | C₅-C₁₂ | 1.3E−02 | 10.8 | 20.7 |
| 8 | MFI-1 | 500 | 17 | 50 | 19 | 6.5 | 92 | C₅-C₈ | 2.4E−02 | 10.2 | 13.1 |
| 9 | MFI-1 | 500 | 16 | 49 | 21 | 6.8 | 93 | C₅-C₈ | 1.9E−02 | 9.6 | 12.7 |
| 10 | MFI-2 | 500 | 18 | 38 | 21 | 6.1 | 83 | C₅-C₈, arom. | 2.3E−02 | 9.1 | 12.5 |
| 11 | MOR-3 | 500 | 15 | 49 | 21 | 7.1 | 92 | C₅-C₈ | 1.4E−02 | 9.0 | 12.0 |
| 12 | BEA-1 | 500 | 5 | 33 | 27 | 5.0 | 70 | C₅-C₁₆ | 1.3E−02 | 7.5 | 13.0 |
| 13 | MFI-3 | 500 | 17 | 50 | 17 | 9.0 | 93 | C₅-C₈, arom. | 1.8E−02 | 7.4 | 9.4 |
| 14 | MFI-6 | 500 | 20 | 43 | 11 | 8.6 | 82 | C₅-C₈ | — | 7.3 | 8.5 |
| 15 | Y-3 | 500 | 5 | 32 | 26 | 5.1 | 68 | C₅-C₁₂, arom. | 1.7E−02 | 7.2 | 12.2 |
| 16 | MOR-1 | 500 | 16 | 49 | 19 | 9.2 | 93 | C₅-C₈, arom. | 1.8E−02 | 7.1 | 9.1 |
| 17 | Y-2 | 500 | 3 | 28 | 26 | 4.9 | 63 | C₅-C₁₂, arom. | 1.8E−02 | 6.4 | 11.7 |
| 18 | Y-1 | 500 | 3 | 31 | 31 | 5.3 | 70 | C₅-C₁₂, arom. | 4.2E−02 | 6.3 | 12.2 |
| 19 | Y-3 | 500 | 5 | 33 | 25 | 6.3 | 69 | C₅-C₁₆ | 1.5E−02 | 6.0 | 10.0 |
| 20 | MFI-1 | 600 | 26 | 46 | 11 | 13.3 | 96 | C₅-C₈ | 1.1E−02 | 5.4 | 6.2 |
| 21 | BEA-4 | 500 | 6 | 37 | 31 | 8.9 | 83 | C₅-C₈ | 1.2E−02 | 4.9 | 8.4 |
| 22 | BEA-2 | 500 | 10 | 43 | 25 | 11.6 | 90 | C₅-C₈ | 1.7E−02 | 4.5 | 6.7 |
| 23 | BEA-1 | 500 | 5 | 18 | 12 | 5.4 | 40 | C₅-C₁₆ | 1.1E−02 | 4.3 | 6.5 |
| 24 | Y-1 | 300 | 1 | 8 | 11 | 2.6 | 23 | C₅-C₁₂ | 3.6E−02 | 3.7 | 8.0 |
| 25 | BEA-3 | 500 | 12 | 43 | 22 | 14.7 | 92 | C₅-C₈, arom. | 1.8E−02 | 3.7 | 5.3 |

¹arom. = aromatics other than BTX

### Examples 26-50-PP

Table 3 shows the experimental results using the listed zeolite catalysts to degrade PP at the listed cracking temperature (T_{cr}). Moplen HP522H PP, available from LyondellBasell Industries, was used as a proxy for PP recyclate. The products of catalytic pyrolysis of PP samples are reported as weight percentages of ethylene (C₂⁼), propylene (C₃⁼), mixed butanes and butenes (C₄), and benzene/toluene/xylene (BTX). Selectivity sum is the combined total amount of forgoing products as a weight percent of all pyrolysis products. “Other products” reports the composition of the balance of the pyrolysis product over the selectivity sum.

Inventive Examples 26-29 show that small pore sized zeolites CHA-1, CHA-2, CHA-3, and ERI-1 produce a higher ratio of light olefins (C₂⁼+C₃⁼) to aromatics BTX than comparative Examples 30-50 using medium or large pore zeolites. The small pore size zeolites consistently show a higher ratio of light olefins (C₂⁼+C₃⁼) to aromatics BTX regardless of polymer being degraded, thus suggesting these zeolites would also outperform medium or large pore zeolites for use with HDPE/PP mixtures.

TABLE 3
Polypropylene (PP)

| | | | Selectivity (wt %) | | | | | | Waste/ | | |
|---|---|---|---|---|---|---|---|---|---|---|---|
| | Zeolite | T_{cr} | | | | | Sel. | Other | Cat | (C₂⁼ + C₃⁼) | (C₂⁼to C₄) |
| Ex. | Catalyst | (° C.) | C₂⁼ | C₃⁼ | C₄ | BTX | Sum. | products¹ | (m/m) | BTX | BTX |
| 26 | ERI-1 | 500 | 7 | 38 | 26 | 1.6 | 73 | C₅-C₁₂ | 1.1E−02 | 27.3 | 43.2 |
| 27 | CHA-3 | 500 | 11 | 34 | 22 | 2.1 | 69 | C₅-C₈ | 1.4E−02 | 21.8 | 32.3 |
| 28 | CHA-2 | 500 | 7 | 31 | 22 | 2.3 | 62 | C₅-C₁₂ | 9.6E−03 | 16.8 | 26.4 |
| 29 | CHA-1 | 500 | 2 | 25 | 22 | 2.1 | 52 | C₅-C₁₂ | 6.5E−02 | 12.8 | 23.5 |
| 30 | MOR-2 | 500 | 5 | 38 | 30 | 3.5 | 77 | C₅-C₈ | 1.0E−02 | 12.3 | 21.0 |
| 31 | BEA-5 | 500 | 2 | 33 | 30 | 3.2 | 68 | C₅-C₈ | 1.4E−02 | 11.2 | 20.6 |
| 32 | MFI-1 | 500 | 20 | 46 | 17 | 7.1 | 91 | C₅-C₈ | 2.7E−02 | 9.5 | 11.8 |
| 33 | MFI-2 | 500 | 21 | 46 | 15 | 7.3 | 90 | C₅-C₈ | 2.4E−02 | 9.2 | 11.3 |
| 34 | MFI-3 | 500 | 18 | 48 | 16 | 7.8 | 90 | C₅-C₈ | 1.4E−02 | 8.5 | 10.5 |
| 35 | MFI-1 | 500 | 16 | 46 | 18 | 7.4 | 88 | C₅-C₈ | 1.5E−02 | 8.3 | 10.8 |
| 36 | Y-2 | 500 | 1 | 31 | 27 | 4.1 | 64 | C₅-C₈ | 1.9E−02 | 7.8 | 14.4 |
| 37 | Y-3 | 500 | 3 | 36 | 27 | 5.0 | 71 | C₅-C₁₂, arom. | 1.8E−02 | 7.8 | 13.2 |
| 38 | MFI-2 | 500 | 17 | 37 | 20 | 7.0 | 81 | C₅-C₈, arom. | 2.3E−02 | 7.7 | 10.6 |
| 39 | Y-1 | 500 | 3 | 33 | 27 | 5.2 | 68 | C₅-C₈, arom. | 4.1E−02 | 7.6 | 13.2 |
| 40 | BEA-1 | 500 | 4 | 36 | 28 | 4.8 | 73 | C₅-C₈ | 1.3E−02 | 7.6 | 13.0 |
| 41 | MOR-1 | 500 | 17 | 44 | 19 | 8.8 | 89 | C₅-C₈, arom. | 1.4E−02 | 7.0 | 9.1 |
| 42 | MOR-3 | 500 | 18 | 43 | 19 | 8.9 | 89 | C₅-C₈, arom. | 1.8E−02 | 6.8 | 9.0 |
| 43 | Y-1 | 400 | 2 | 22 | 27 | 3.7 | 54 | C₅-C₈, arom. | 1.8E−02 | 6.5 | 13.9 |
| 44 | Y-1 | 300 | 2 | 17 | 21 | 3.3 | 44 | C₅-C₈, arom. | 4.4E−02 | 5.9 | 12.3 |
| 45 | MFI-1 | 600 | 22 | 47 | 11 | 12.2 | 92 | C₅-C₈ | 1.3E−02 | 5.7 | 6.6 |
| 46 | BEA-2 | 500 | 8 | 44 | 24 | 9.4 | 86 | C₅-C₈, arom. | 1.5E−02 | 5.6 | 8.1 |
| 47 | BEA-4 | 500 | 5 | 38 | 29 | 7.9 | 79 | C₅-C₈ | 1.1E−02 | 5.4 | 9.0 |
| 48 | MFI-6 | 500 | 17 | 37 | 21 | 10.2 | 86 | C₅-C₈ | — | 5.3 | 7.4 |
| 49 | BEA-1 | 500 | 3 | 20 | 12 | 5.5 | 41 | C₅-C₁₂ | 1.7E−02 | 4.3 | 6.4 |
| 50 | BEA-3 | 500 | 10 | 43 | 21 | 12.5 | 87 | C₅-C₈, arom. | 1.5E−02 | 4.3 | 6.0 |

¹arom. = aromatics other than BTX

### Examples 51-68-TPG

Table 4 shows the experimental results using the listed zeolite catalysts to degrade TPG at the listed cracking temperature (T_{cr}). Solvent grade TPG, available from LyondellBasell, was used as a proxy for polyol recyclate. The products of catalytic pyrolysis of TPG samples are reported as weight percentages of ethylene (C₂⁼), propylene (C₃⁼), mixed butanes and butenes (C₄), and benzene/toluene/xylene (BTX). Selectivity sum is the combined total amount of forgoing products as a weight percent of all pyrolysis products. Other products reports the composition of the balance of the pyrolysis product over the selectivity sum.

Inventive Examples 51-54 show that small pore sized zeolites CHA-1, CHA-2, CHA-3, and ERI-1 produce a higher ratio of light olefins (C₂⁼+C₃⁼) to aromatics BTX than comparative Examples 55-68 using medium or large pore zeolites. The small pore size zeolites consistently show a higher ratio of light olefins (C₂+C₃⁼) to aromatics BTX regardless of whether the material being degraded is a polymer or a polyol, thus suggesting these zeolites would also outperform medium or large pore zeolites for use with mixtures comprising polymers and polyols.

TABLE 4
TriPropylene Glycol (TPG)

| | | | Selectivity (wt %) | | | | | | | |
|---|---|---|---|---|---|---|---|---|---|---|
| | Zeolite | T_{cr} | | | | | Sel. | Other | (C₂⁼ + C₃⁼) | (C₂⁼to C₄) |
| Ex. | Catalyst | (° C.) | C₂⁼ | C₃⁼ | C₄ | BTX | Sum. | products^{1,2} | BTX | BTX |
| 51 | CHA-1 | 500 | 26 | 30 | 12 | 0 | 67 | C₅-C₆, oxy. | — | — |
| 52 | CHA-2 | 500 | 51 | 30 | 10 | 0 | 91 | oxy. | — | — |
| 53 | CHA-3 | 500 | 57 | 32 | 6 | 0 | 96 | oxy. | — | — |
| 54 | ERI-1 | 500 | 54 | 33 | 9 | 0.9 | 97 | C₅-C₆ | 102.3 | 113.4 |
| 55 | MOR-3 | 500 | 30 | 34 | 9 | 23 | 96 | arom. | 2.7 | 3.1 |
| 56 | BEA-5 | 500 | 8 | 24 | 13 | 13 | 59 | C₅-C₆, oxy., arom. | 2.5 | 3.5 |
| 57 | Y-2 | 500 | 5 | 35 | 19 | 17 | 75 | C₅-C₆, oxy., arom. | 2.2 | 3.3 |
| 58 | BEA-1 | 500 | 4 | 19 | 5 | 11 | 38 | oxy. | 2.2 | 2.7 |
| 59 | MOR-1 | 500 | 23 | 32 | 8 | 29 | 91 | arom. | 1.9 | 2.2 |
| 60 | BEA-1 | 500 | 11 | 32 | 10 | 24 | 77 | oxy., arom. | 1.8 | 2.2 |
| 61 | Y-3 | 500 | 7 | 30 | 21 | 22 | 80 | oxy., arom. | 1.7 | 2.6 |
| 62 | BEA-4 | 500 | 9 | 31 | 11 | 23 | 74 | oxy., arom. | 1.7 | 2.2 |
| 63 | BEA-2 | 500 | 14 | 35 | 9 | 31 | 89 | arom. | 1.6 | 1.9 |
| 64 | MFI-2 | 500 | 17 | 16 | 10 | 22 | 65 | C₅-C₁₆, oxy., arom. | 1.5 | 1.9 |
| 65 | BEA-3 | 500 | 15 | 32 | 9 | 34 | 90 | arom. | 1.4 | 1.6 |
| 66 | MFI-3 | 500 | 25 | 25 | 6 | 39 | 93 | arom. | 1.3 | 1.4 |
| 67 | MFI-1 | 400 | 12 | 23 | 12 | 33 | 80 | oxy., arom. | 1.0 | 1.4 |
| 68 | MFI-6 | 500 | 15 | 21 | 11 | 37 | 84 | C₅-C₁₂, oxy., arom. | 1.0 | 1.3 |

¹arom. = aromatics other than BTX
²oxy. = oxygenates

### Examples 69-70-Soybean Oil

Table 5 shows the experimental results using the listed zeolite catalysts to degrade soybean oil at the listed cracking temperature (Ter). Great Value™ brand soybean oil available from Walmart, was used as a proxy for polyol recyclate. The products of catalytic pyrolysis of soybean oil samples are reported as weight percentages of ethylene (C₂⁼), propylene (C₃⁼), mixed butanes and butenes (C₄), and benzene/toluene/xylene (BTX). Selectivity sum is the combined total amount of forgoing products as a weight percent of all pyrolysis products. Other products reports the composition of the balance of the pyrolysis product over the selectivity sum.

Inventive Example 69 shows that small pore sized zeolite ERI-1 produces a higher ratio of light olefins (C₂+C₃⁼) to aromatics BTX (25.6:7.3) than comparative Example 70 using medium pore zeolite MFI-1 (38.6:27.6). The small pore size zeolites consistently show a higher ratio of light olefins (C₂-+C₃⁼) to aromatics BTX regardless of whether the material being degraded is a polymer, a polyol, or soybean oil, thus suggesting these zeolites would also outperform medium or large pore zeolites for use with mixtures comprising a polymer, a polyol, and/or a biomass composition such as soybean oil.

TABLE 5
Soybean oil

| | | | Selectivity (wt %) | | | | | | Waste/ | | |
|---|---|---|---|---|---|---|---|---|---|---|---|
| | Zeolite | T_{cr} | | | | | Sel. | Other | Cat | (C₂⁼+ C₃⁼) | (C₂⁼to C₄) |
| Ex. | Catalyst | (° C.) | C₂⁼ | C₃⁼ | C₄ | BTX | Sum. | products¹ | (m/m) | BTX | BTX |
| 69 | ERI-1 | 500 | 6.8 | 18.8 | 13.9 | 7.3 | 47 | C₅-C₁₅, arom. | 8.9E−02 | 3.5 | 5.4 |
| 70 | MFI-1 | 500 | 11.8 | 26.8 | 15.0 | 27.6 | 81 | C₅-C₈, arom. | 8.9E−02 | 1.4 | 1.9 |

¹arom. = aromatics other than BTX

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, methods, and/or steps described in the specification. As one of the ordinary skill in the art will readily appreciate from the disclosure, processes, machines, means, methods, and/or steps, presently existing or later to be developed that perform substantially the same function or achieve substantially the same result as the corresponding embodiments described herein, may be utilized according to the present disclosure. Accordingly, the appended claims are intended to include within their scope such processes, machines, means, methods, and/or steps.

What is claimed is:

1. A process comprising:
contacting a hydrocarbonaceous feedstock with a zeolite catalyst having a pore size ranging from 0.30 nanometers to 0.50 nanometers in a reaction zone to produce a product stream comprising an olefin component and an aromatic component, wherein:
the olefin component comprises ethylene, propylene, or a combination thereof;
the aromatic component comprises benzene, toluene, xylene, or a combination thereof; and
the product stream comprises the aromatic component in an amount less than or equal to 8 wt %, based on the total weight of the product stream.

2. The process of claim 1, wherein a weight ratio of the olefin component to the aromatic component is greater than or equal to 2.0.

3. The process of claim 1, wherein the reaction zone comprises the zeolite catalyst as a fixed catalyst bed or a fluidized catalyst bed.

4. The process of claim 1, further comprising subjecting the product stream to one or more separation processes to recover an olefin product stream comprising at least 80 wt % of the olefin component.

5. The process of claim 1, wherein the contacting step is performed at:
a temperature in the range of from 200° C. to 800° C.;
a pressure in the range of from 5 kPa (absolute) to 10 MPag;
a weight hourly space velocity in the range of from 0.1 hr⁻¹ to 100 hr⁻¹; or
a combination thereof.

6. The process of claim 1, wherein the zeolite catalyst:
comprises 8-member ring pores;
has a silica to alumina ratio (SAR) in the range of from 5 to 35; or
a combination thereof.

7. The process of claim 1, wherein the hydrocarbonaceous feedstock comprises a polymer recyclate.

8. The process of claim 7, wherein the polymer recyclate comprises polyethylene recyclate, polypropylene recyclate, or a combination thereof.

9. The process of claim 1, wherein the hydrocarbonaceous feedstock comprises one or more polyols.

10. The process of claim 9, wherein the one or more polyols comprise a trihydric alcohol.

11. The process of claim 1, wherein the hydrocarbonaceous feedstock comprises a biomass composition.

12. The process of claim 11, wherein the biomass composition comprises a glyceride composition.

13. The process of claim 12, wherein the glyceride composition comprises a triglyceride.

14. The process of claim 1, further comprising:
contacting a hydrocarbonaceous waste stream with a poison mitigation compound in a guard zone to produce a treated hydrocarbonaceous waste stream; and
withdrawing the treated hydrocarbonaceous waste stream as the hydrocarbonaceous feed stream;
wherein:
the hydrocarbonaceous waste stream comprises a first hydrocarbonaceous component and a first contaminant component;
the treated hydrocarbonaceous waste stream comprises the first hydrocarbonaceous component and a second contaminant component;
the first contaminant component would deactivate the zeolite catalyst at a first deactivation rate;
the second contaminant component would deactivate the zeolite catalyst at a second deactivation rate; and
the first deactivation rate is greater than the second deactivation rate.

15. The process of claim 1, further comprising:
thermally pyrolyzing a hydrocarbonaceous waste stream to produce a first pyrolysis product in a thermal pyrolysis reaction zone;
separating the first pyrolysis product into a first pyrolysis light fraction and a first pyrolysis heavy fraction in a separation reaction zone; and
withdrawing the first pyrolysis light fraction as the hydrocarbonaceous feed stream.

16. The process of claim 15, further comprising:
adding at least a portion of the first pyrolysis heavy fraction as additional feed to the thermal pyrolysis reaction zone;
removing at least a portion of the first pyrolysis heavy fraction from the process; or
a combination thereof.

17. The process of claim 1, further comprising:
contacting a hydrocarbonaceous waste stream with a poison mitigation compound in a guard zone to produce a treated hydrocarbonaceous waste stream;
thermally pyrolyzing the treated hydrocarbonaceous waste stream to produce a first pyrolysis product in a thermal pyrolysis reaction zone;
separating the first pyrolysis product into a first pyrolysis light fraction and a first pyrolysis heavy fraction in a separation reaction zone; and
withdrawing the first pyrolysis light fraction as the hydrocarbonaceous feed stream;
wherein:
the hydrocarbonaceous waste stream comprises a first hydrocarbonaceous component and a first contaminant component;
the treated hydrocarbonaceous waste stream comprises the first hydrocarbonaceous component and a second contaminant component;
the first contaminant component would deactivate the zeolite catalyst at a first deactivation rate;
the second contaminant component would deactivate the zeolite catalyst at a second deactivation rate; and
the first deactivation rate is greater than the second deactivation rate.

18. The process of claim 17, further comprising:
adding at least a portion of the first pyrolysis heavy fraction as additional feed to the thermal pyrolysis reaction zone;
removing at least a portion of the first pyrolysis heavy fraction from the process; or
a combination thereof.

19. The process of claim 1, further comprising:
thermally pyrolyzing a hydrocarbonaceous waste stream to produce a first pyrolysis product in a thermal pyrolysis reaction zone;
separating the first pyrolysis product into a first pyrolysis light fraction and a first pyrolysis heavy fraction in a separation reaction zone; and
contacting the first pyrolysis light fraction with a poison mitigation compound in a guard zone to produce a treated first pyrolysis light fraction; and
withdrawing the treated first pyrolysis light fraction as the hydrocarbonaceous feed stream;
wherein:
the first pyrolysis light fraction comprises a first hydrocarbonaceous component and a first contaminant component;
the treated first pyrolysis light fraction comprises the first hydrocarbonaceous component and a second contaminant component;
the first contaminant component would deactivate the zeolite catalyst at a first deactivation rate;
the second contaminant component would deactivate the zeolite catalyst at a second deactivation rate; and
the first deactivation rate is greater than the second deactivation rate.

20. The process of claim 19, further comprising:
adding at least a portion of the first pyrolysis heavy fraction as additional feed to the thermal pyrolysis reaction zone;
removing at least a portion of the first pyrolysis heavy fraction from the process; or
a combination thereof.
